(19) United States
(12) Patent Application Publication
(10) Pub. No.: US 2006/0206014 A1
(43) Pub. Date: Sep. 14, 2006

(54) Ear probe particularly for measuring various physiological conditions particularly blood pressure, temperature and/or respiration

(75) Inventor: Arie Ariav, Doar-Na Hof Ashkelon (IL)

Correspondence Address: Martin D. Moynihan;PRTSI, Inc., P.O. Box 16446, Arlington, VA, 22215, US

(73) Assignee: Nexense Ltd.

(21) Appl. No.: 11/373,280

(22) Filed: Mar. 13, 2006

(30) Foreign Application Priority Data
Mar. 13, 2005 (IL) 167401

(51) Int. Cl. A61B 5/00; A61B 5/02; A61B 5/08

(52) U.S. Cl. 600/301; 600/549; 600/485; 600/529; 128/905

(57) ABSTRACT

An ear probe for detecting and/or measuring at least one predetermined physiological condition of a subject, includes: an earplug shaped and dimensioned for insertion into an ear of the subject; and a sensor carried by the earplug for sensing the predetermined physiological condition of the subject; the sensor including: an acoustical transmitter and an acoustical receiver spaced from the transmitter and defining an acoustical channel therebetween; and a processor for monitoring changes in the transit time, caused by the predetermined physiological condition, of an acoustical wave transmitted from the transmitter to the receiver through the acoustical channel.

### RELATED APPLICATION

This application includes subject matter, and claims the priority date of Israel Patent Application No 167401, filed on Mar. 13, 2005, the contents of which are incorporated herein by reference.

### FIELD AND BACKGROUND OF THE INVENTION

The present invention relates to an ear probe for detecting and/or measuring various physiological conditions of a subject. The invention is particularly useful for measuring body temperature, blood pressure and/or respiration of a subject, and is therefore described below with respect to such applications.

The accurate measurement of various physiological conditions, particularly body temperature and blood pressure, is frequently necessary or even critical in many medical situations. The most accurate way of making such measurements is by inserting an arterial catheter into the subject's artery; but such a method is highly invasive, requires a high degree of expertise, and runs a number of risks, e.g., infection, occlusion of an artery, or damage to an artery. Such highly invasive techniques are therefore used only when absolutely necessary, e.g., during major operating procedures or during intensive care where a precise blood-pressure measurement and/or temperature measurement is continuously needed.

Accordingly, many non-invasive procedures for making measurements of various physiological conditions are generally used even though less accurate compared to an invasive procedure. Several ear probes have been developed for this purpose since relatively accurate temperature and pressure measurements can be made non-invasively from the carotid artery which is close to the ear canal. Examples of ear probes for measuring temperature are described in U.S. Pat. Nos. 6,556,852 and 6,773,405, and for measuring blood pressure are described in U.S. Pat. Nos. 5,237,997 and 6,004,274. However efforts are continually being made to improve the sensitivity and accuracy of such ear probes.

### OBJECT AND BRIEF SUMMARY OF THE PRESENT INVENTION

An object of the present invention is to provide an ear probe which enables the accurate, non-invasive measurement of various physiological conditions, particularly temperature and/or blood pressure, in addition to various blood-pressure and respiration parameters, as will be described more particularly below.

According to one aspect of the present invention, there is provided an ear probe for detecting and/or measuring at least one predetermined physiological condition of a subject, comprising: an ear plug shaped and dimensioned for insertion into an ear of the subject; and a sensor carried by the earplug for sensing the predetermined physiological condition of the subject; the sensor including an acoustical transmitter and an acoustical receiver spaced from the transmitter and defining an acoustical channel therebetween; and a processor for monitoring changes in the transit time, caused by the predetermined physiological condition, of an acoustical wave transmitted from the transmitter to the receiver through the acoustical channel.

As indicated above, the invention is particularly useful for detecting and/or measuring body temperature and/or blood pressure, as well as various blood-pressure and respiration parameters, and is therefore described below with respect to such applications.

Thus, the present invention also utilizes the ear, and particularly the carotid artery accessible non-invasively from the ear canal, for sensing the respective physiological conditions to be measured, as in the prior art. However, instead of using conventional techniques, such as thermistors for making temperature measurements and strain gauges for making blood-pressure measurements as in the prior art, the present invention uses instead an acoustical technique, wherein what is measured is the influence of the respective condition on the transit time of an acoustical wave transmitted through an acoustical channel from a transmitter to a receiver. Thus, the condition being measured (e.g. temperature, pressure, etc.) influences the transit velocity and/or the transit distance of such an acoustical wave, and therefore by precisely measuring the transit time of such an acoustical wave, the respective physiological condition can be precisely measured.

In the preferred embodiments of the invention described below, the processor measures changes in transit time in accordance with the method described in U.S. Pat. No. 6,621,278 (Israel Patent 129,651) and International Application PCT/IL2004/000138, International Publication No WO2004/072658, published Aug. 26, 2004, both assigned to the same assignee as the present application, the contents of which are expressly incorporated herein by reference. As described therein, the processor measures such changes in the transit time by controlling the frequency of the transmitter to produce and maintain a whole integer number of waves in a loop including the acoustical channel irrespective of changes in the physiological condition being measured. This is done by detecting a fiducial point of each wave received by the receiver in the respective acoustical channel, and utilizing the detected fiducial point to trigger the transmission of the next wave by the transmitter in the respective acoustical channel.

In the preferred embodiments of the invention described below, one measured physiological condition is the temperature of the blood in the subject's carotid artery as such a temperature measurement is considered to be a very accurate representation of the subject's body temperature.

A second physiological condition measured in accordance with the present invention as described below is the blood pressure in the subject's carotid artery. Such a pressure measurement is also considered to be a very accurate representation of the patient's blood pressure. Measuring the blood pressure in the carotid artery in accordance with the present invention enables a large number of cardiac conditions to be detected and/or measured, including heart rate, systolic pressure, diastolic pressure, mean arterial pressure, continuous blood pressure, stroke volume and cardiac output.

A third physiological condition accurately measured by the described probe is a respiratory condition of the subject, such as respiration rate, respiration volume, sleep apnea and snoring. As further described below, the ear probe could also be used for measuring various body movements, such as walking or running by the subject.

In the described preferred embodiments, the earplug includes at least two such sensors for accurately measuring two or more physiological conditions of the subject.

According to another aspect of the present invention, there is provided an ear probe for detecting and/or measuring a plurality of physiological conditions of a subject, comprising: an earplug shaped and dimensioned for insertion into the ear canal of the subject; a temperature sensor carried by the earplug for sensing the temperature of the blond in the subject's carotid artery; and a pressure sensor carried by the earplug for sensing changes in, blood pressure in the carotid artery of the subject.

According to further features in the described preferred embodiments, the earplug is of a flexible material shaped and dimensioned to seal the ear canal and to produce a sealed air chamber between the end of the earplug and the subject's ear-drum. The temperature sensor is exposed to the sealed air chamber to sense the temperature thereof; and the pressure sensor is also exposed to the sealed air chamber to sense changes in the pressure therein.

In one described preferred embodiment, the pressure sensor includes a body of elastomeric material which is compressed by the pressure in the sealed air chamber. In a second described embodiment the pressure sensor includes a membrane which is deformed by the pressure in the sealed air chamber. A further embodiment is described wherein the ear probe includes an external pump which controllably pressurizes the sealed air chamber, and thereby the carotid artery, to enable blood pressure measurements to be made according to the oscillometric technique or other techniques involving the controlled application of pressure to an artery.

As will be described more particularly below, an ear probe constructed in accordance with one or more of the foregoing features enables highly accurate measurements of various physiological conditions, particularly body temperature, blood pressure and various respiratory activities, to be made in a convenient and non-invasive manner.

Further features and advantages of the invention will be apparent from the description below.

### BRIEF DESCRIPTION OF THE DRAWINGS

The invention is herein described, by way of example only, with reference to the accompanying drawings, wherein:

FIG. 1 illustrates one form of ear probe constructed in accordance with the present invention;

FIG. 2 is an enlarged fragmentary view more particularly illustrating the sensors in the ear probe of FIG. 1;

FIG. 3 illustrates a preferred system for measuring the transit time of the acoustical waves in each of the sensors of FIGS. 1 and 2;

FIG. 4 illustrates another ear probe constructed in accordance with the present invention;

FIG. 5 more particularly illustrates the pressure sensor in the ear probe of FIG. 4; and

FIG. 6 illustrates a further ear probe constructed in accordance with the present invention.

It is to be understood that the foregoing drawings, and the description below, are provided primarily for purposes of facilitating understanding the conceptual aspects of the invention and various possible embodiments thereof including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

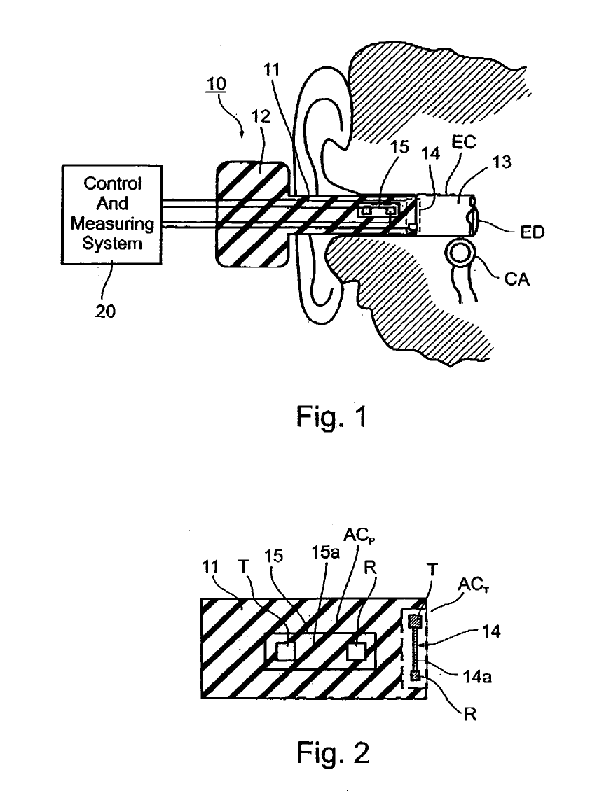

The Embodiment of FIGS. 1 and 2

FIG. 1 illustrates an ear probe, therein generally designated 10, constructed in accordance with the present invention, for detecting and/or measuring a plurality of different physiological conditions of the subject. In this case, ear probe 10 is capable of detecting and/or measuring body temperature of the subject, cardiac activity of the subject, respiratory activity of the subject, and other body movements of the subject.

Thus, ear probe 10 illustrated in FIG. 1 includes an ear plug 11 of a resilient material, such as an elastomeric material, shaped and dimensioned for insertion into the ear canal of the subject. Earplug 11 includes an enlarged head 12 extending externally of the subject's ear to facilitate insertion and removal of the ear probe.

Earplug 11 is shaped and dimensioned to seal the subject's ear canal EC and to produce a sealed air chamber 13 between the end of the earplug and the subject's ear-drum. ED. The inner end of earplug 11 carries a temperature sensor 14 which is directly exposed to the sealed air chamber 13 and thereby measures its temperature. As shown in FIG. 1, the sealed air chamber 13 borders the carotid artery CA, and therefore its temperature will be substantially the same as that of the blood in the carotid artery.

The pulsatile pressure of the blood flowing through the carotid artery will also be applied to the sealed air chamber 13. This pressure is sensed by a pressure sensor 15 also carried at the inner end of the earplug.

As indicated earlier, each of the sensors 14 and 15 is of the acoustical type. Each includes an acoustical channel having an acoustical transmitter T (FIG. 2) and an acoustical receiver R through which propagates an acoustical wave at a transit velocity, and/or for a transit distance, which varies according to the condition being sensed by the respective sensor. Therefore, by precisely measuring the transit time of the acoustical wave through the respective acoustical channel, a measurement is produced of the physiological condition sensed by the respective sensor.

In FIGS. 1 and 2, the temperature sensor 14 at the inner tip of earplug 11 is defined by the acoustical channel AC_{T}. It is constituted of a strip or film 14a of a material having high thermal conductivity, such as metal, between its respective transmitter T and receiver R. Since strip 14a is exposed to the temperature within the sealed air chamber 13, the latter temperature will influence the transit velocity, and also the transit distance (by elongation), of the acoustical waves transmitted in that channel, and therefore the measurement of the transit time of such acoustical waves will also represent a measurement of the temperature within the sealed air chamber 13.

The pressure sensor 15 includes a transmission channel (AC_{P}) defined by the portion 15a of the material of earplug 11 between its transmitter T and receiver R. Portion 15a of earplug 11, or the complete earplug, is preferably made of a soft, deformable, elastomeric material having high transmissivity and low attenuation with respect to acoustical waves. Examples of materials that can be used for this purpose are described in the above-cited International Application No. PCT/IL2004/000138.

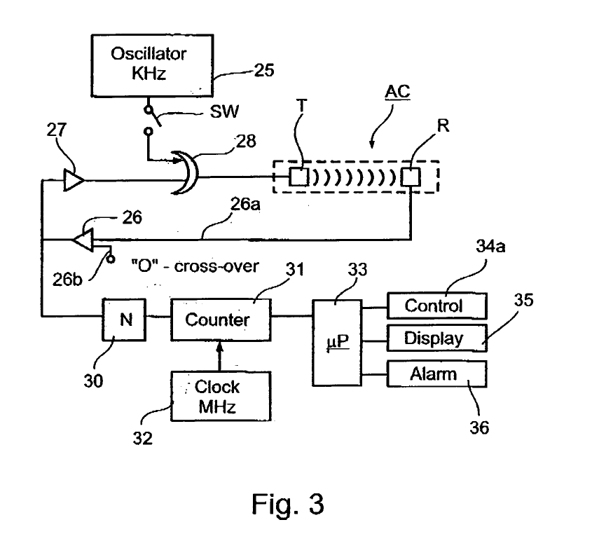

The transmitters and receivers in each of the two acoustical channels AC_{T} and AC_{P} are controlled by a control and measuring system 20 in the form of a microprocessor (FIG. 1). Microprocessor 20 is effective to measure changes in the transit time of acoustical waves in each of the transmission channels AC_{T} and AC_{P}. As indicated earlier, and as more particularly described below with respect to FIG. 3, this is done by, controlling the frequency of the transmitter in each of the acoustical channels to produce and maintain a whole integer number of waves in the respective channel irrespective of changes in the condition being measured, namely temperature in channel AC_{T} and pressure in charnel AC_{P}.

The Control and Measuring System

FIG. 3 more particularly illustrates the control and measuring system for each of the acoustical channels AC_{T} and AC_{P}.

Initially, the transmitter T in each acoustical channel, therein designated AC, is activated by an oscillator 25 under the control of a switch SW until the acoustical waves are received by the receiver R in the respective channel. Once these waves are received, switch SW is opened, so that the received waves are thereafter used for triggering the transmitter T in the respective channel.

Thus, as shown in FIG. 3, the receiver R in the respective channel produces an output which is fed to a comparator 26 via its input line 26a. Comparator 26 includes a second input 26b connected to a predetermined bias so as to detect a predetermined fiducial or reference point in the received signal. In the example illustrated in FIG. 3, this predetermined fiducial point is the “0” cross-over point of the signal outputted from receiver R; hence, input 26b is at a “0” bias. It will be appreciated, however, that other reference points could be used as a fiducial point, such as the maximum peaks, the minimum peaks, or the leading edges of the output signal from the receiver R.

The output from comparator 26 is fed to a monostable oscillator 27 which is triggered to produce an amplified output signal at each fiducial point in the signal from the receiver R. The signals from monostable oscillator 27 are fed via an OR-gate 28 to trigger the transmitter T in the respective acoustical channel AC.

It will thus be seen that transmitter T in each acoustical charnel is activated at a frequency such as to maintain the number of waves in the respective channels as a whole integer irrespective of changes in the condition (e.g. temperature, pressure) being monitored. This produces a precise measurement of the changes in transit time of the waves in the respective channel, and thereby of the condition influencing the change in the transit time, whether resulting from a change in the transit velocity, and/or the transit distance, of the wave through the respective channel.

FIG. 3 also illustrates a circuit for accumulating small changes in frequency over a large time interval. Thus, the signals outputted from comparator 26, used for controlling the triggering of transmitter T, are also fed to a counter 30 to be counted N times, and the output is fed to another counter 31 controlled by clock 32. Counter 31 produces an output to microprocessor 33 which performs a computation, according to the parameter or condition to be measured, in this case the temperature measured by sensor 14 including its acoustical channel AC_{T}, and the pressure as measured by sensor 15 including its acoustical channel AC_{P}. As shown in FIG. 3, microprocessor 33 of each sensor 14, 15 produces an output to a control unit 34, to a display unit 35, and/or to an alarm unit 36.

Further details of the construction, use, and other possible applications of the system illustrated in FIG. 3 are set forth in the above-cited U.S. Pat. No. 6,621,278 and International Patent Application No. PCT/IL2004/000138.

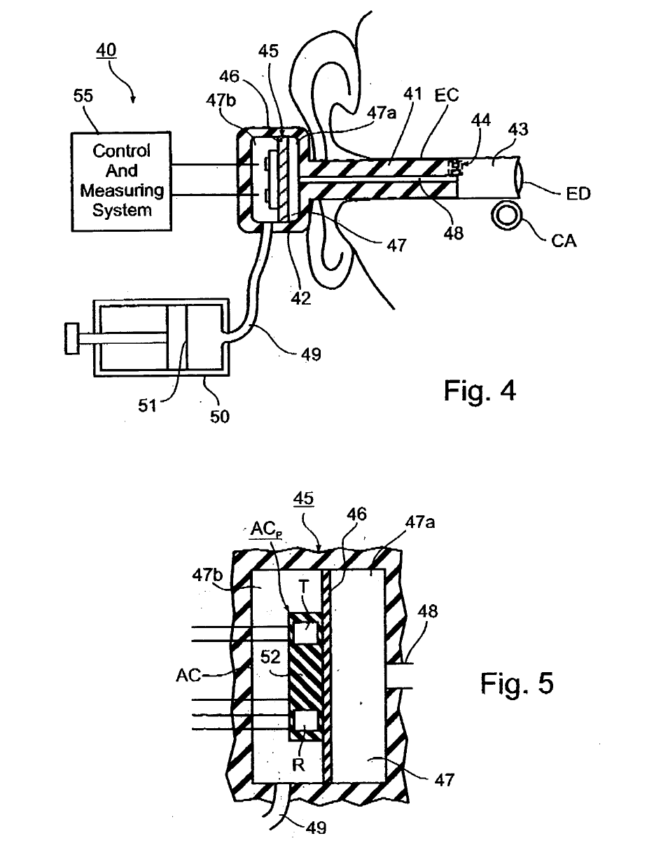

The Ear Probe of FIGS. 4 and 5

FIGS. 4 and 5 illustrate an ear probe also including a temperature sensor and a pressure sensor, as in FIGS. 1-3, but of a different construction particularly with respect to the pressure sensor.

Thus, as shown in FIG. 4, the ear probe, therein generally designated 40, also includes an earplug 41 shaped and dimensioned for insertion into the ear canal EC of the subject and formed with an enlarged head 42 extending externally of the subject's ear. Earplug 41 is made of a material, such as an elastomeric material, which effectively seals the ear canal EC and produces a sealed air chamber 43 between the end of the earplug and the subject's ear-drum ED. As described above with respect to FIG. 1, sealed air chamber 43 is proximate to the subject's carotid artery CA, such as to reflect both the temperature of the blood within that artery, and the changes in pressure produced by the pulsatile flow of the blood through that artery.

The inner tip of earplug 41 carries a temperature sensor 44, corresponding to temperature sensor 14 in FIGS. 1 and 2, to sense the temperature of the air within chamber 43. Temperature sensor 44 is also in the form of an acoustical channel, corresponding to acoustical channel ACT in FIGS. 1 and 2, and also includes a transmitter, a receiver, and a strip of thermally-conductive material (e.g. metal) connecting the two, as described above with respect to FIGS. 1 and 2.

Pressure sensor 45, however, is of a substantially different construction from pressure sensor 15 in the ear probe of FIGS. 1 and 2. Thus, as shown in FIG. 4, pressure sensor 45 is incorporated in the enlarged head 42 of the earplug, rather than in the body of the earplug as in FIGS. 1 and 2. Moreover, instead of being in the form of a compressible elastomeric material, as in FIGS. 1 and 2, pressure sensor 45 in FIG. 4 is in the form of a deformable partition or membrane 46 disposed in a pressure-sensing chamber 47, dividing chamber 47 into two sections 47a, 47b.

FIG. 5 more particularly illustrates the construction of pressure-sensitive chamber 47, the deformable membrane 46 dividing the chamber into the two sections or compartments 47a, 47b, and the acoustical channel AC_{P} mounted on membrane 46 for monitoring the deformations of the membrane in accordance with the differential pressure on its opposite sides. Such a differential-pressure type sensor is more particularly described in the above-cited International Patent Application PCT/IL2004/000138.

As shown in FIG. 4, compartment 47a of pressure-sensing chamber 47 communicates with the sealed air chamber 43 near the carotid artery CA by means of a passageway 48 extending through the earplug 41. On the other hand, compartment 47b of pressure-sensing chamber 47 communicates, via a tube 49, with an external syringe 50 having a manually-movable plunger 51. Plunger 51 may be manually moved to initialize or otherwise control the pressure within compartment 47b such that membrane 46 will deform in response to the differential pressure on its opposite faces. The deformations of membrane 46 are thus a measurement of the changes in the pressure within the sealed air chamber 43 as communicated to compartment 47a via passageway 48.

The deformations of membrane 46 in response to the differential pressure on its opposite faces is measured by pressure sensor 45. Thus, as shown in FIG. 5, pressure sensor 45 is also of the acoustical channel type, including an elastomeric strip 52 having a transmitter T at one end, and a receiver R at the opposite end. Transmitter T and receiver R are piezoelectric devices each connected to a power supply via two leads. A fifth lead may be provided for a shielding electrode (not shown) if desired.

It will be seen that the pressure within compartment 47b can be preset, as desired, by manually manipulating plunger 51 of syringe 50, such that membrane 46 will deform in accordance with the pressure in compartment 47a communicating, via passageway 48, with the sealed air chamber 43 at the inner end of the earplug. Deformations of membrane 46 produce corresponding deformations of elastomeric strip 52, and thereby, corresponding changes in the transit distance of the acoustical waves from transmitter T to receiver R. The transit times of the waves through the acoustical channel of elastomeric strip 52 are thus changed in accordance with changes in the pressure within chamber 43.

As pointed out above, the pressure in chamber 43 follows the changes in the blood pressure within the carotid artery CA produced by the pulsatile blood flow therethrough. Accordingly, a measurement of the changes in the transit times of the acoustical waves from transmitter T to the receiver R within the acoustical channel defined by elastomeric strip 52, caused by deformations of membrane 46, will produce a measurement of the changes of blood pressure within the carotid artery CA.

Further details of the construction of pressure sensor 45, as well as possible variations thereof, are set forth in the above-cited International Application PCT/IL2004/000138.

Both the temperature sensor 44 and the pressure sensor 45 in the ear probe 40 of FIG. 4 are controlled by the control and measuring system 55 in the same manner as described above with respect to temperature sensor 14 and pressure sensor 15 in the ear probe of FIGS. 1 and 2 as described with respect to FIG. 3.

It will thus be seen that the sensor illustrated in FIGS. 4 and 5 is capable of high precision measurements of both body temperature and blood pressure. The latter measurements enable various parameters of blood pressure to be measured, including heart rate, systolic pressure, diastolic pressure, mean arterial pressure, continuous blood pressure, stroke volume, and cardiac output.

The pressure sensor in the ear probe of FIGS. 4 and 5 is also capable of sensing and/or measuring various respiratory conditions, including respiration rate, respiration volume, sleep apnea, and snoring. The pressure sensor also can sense other body movements, such as walking and running, and therefore can be used for measuring walking and running distances, velocity, etc.

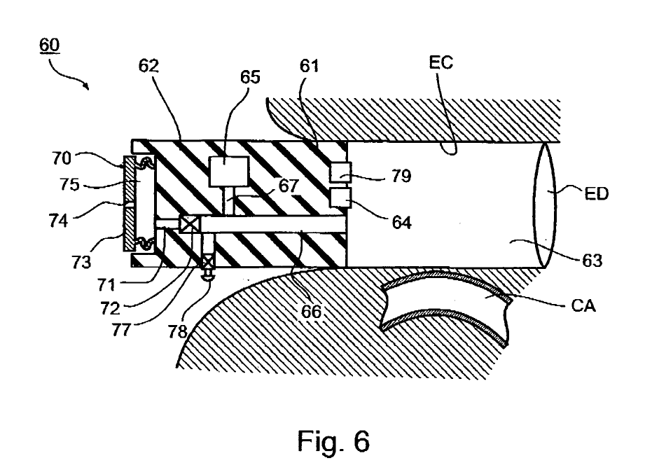

The Ear Probe of FIG. 6

FIG. 6 illustrates an ear probe similar to that illustrated in FIG. 4, except that it is provided with means for pressurizing the sealed air chamber 43 in order to enable the probe to be used for measuring blood pressure, and various parameters of blood pressure measurements, by other known noninvasive methods involving the controlled application of pressure to an artery.

Thus, one known method for non-invasively measuring blood pressure is according to the “oscillometric” technique. The conventional oscillometric technique uses the conventional occluding cuff and pressure sensors for monitoring the pressure inside the occluding cuff to detect pressure oscillations in the artery between the systolic and diastolic arterial pressures. Detecting these pressure oscillations enables various parameters of blood pressure to be determined, including systolic pressure, diastolic pressure, and mean pressure.

The ear probe illustrated in FIG. 6 includes an external pump which may be used controllably to pressurize and depressurize the air sealed chamber, while the pressure sensor within the ear probe measures the pressure oscillations produced as a result in the carotid artery CA.

Thus, the ear probe illustrated in FIG. 6, and therein generally designated 60, also includes an earplug 61 sealingly receivable within the ear canal EC and having a head 62 projecting externally of the subject's ear. In FIG. 6, the external head 62 is shown as approximately of the same diameter as the plug 61, but in most cases it will be of larger diameter to facilitate manipulation of the ear probe and proper location of the earplug within the ear canal with respect to the ear-drum ED.

As in the previously-described embodiments, the earplug 61 is received within the ear canal so as to define a sealed air chamber 63 between the inner tip of the earplug and the subject's ear-drum ED. Earplug 61 carries a temperature sensor 64 at its inner face, and a pressure sensor 65 spaced from its inner face and communicating with the air sealed chamber 63 by passageways 66 and 67. Temperature sensor 64 and pressure sensor 65 may be of the same construction as temperature sensor 14 and pressure sensor 15 in FIGS. 1 and 2, and both sensors may be controlled as described above with respect to FIG. 3.

The external head 62 of ear probe 60 carries a pump 70 having a pressurized output 71 communicating, via one-way valve 72, with passageway 66 leading to the air-sealed chamber 63, as well as with passageway 67 leading to the pressure sensor 65. Pump 70 is a manual pump and includes a depressable finger-piece 73 formed with a vent opening 74. The construction is such that when finger-piece 73 is engaged by the user's finger and is manually depressed, the air within pump chamber 75 is forced via outlet 71 and one-way valve 72, through passageway 66 to thereby pressurize chamber 63; and when finger-piece 73 is released to uncover vent opening 74, one-way valve 72 blocks the reverse flow of air such that pump chamber 75 is refilled with air via the vent opening.

The external head 62 of ear probe 60 includes a further valve 77 communicating with passageway 66. Valve 77 serves two functions: It prevents over-pressurization of the air sealed chamber 63 to prevent possible injury to the ear-drum. It also permits release of the pressure within chamber 63, and for this purpose it may be provided with a manual release element, such as a plunger 78, for controllably opening the valve to permit a controlled or a complete depressurization of chamber 63.

It will thus be seen that pump 70 may be operated to produce the initial overpressurization of chamber 63, and then valve 77 may be operated to reduce this pressure such as to produce pressure oscillations within the carotid artery CA. Such pressure oscillations are detected and measured by pressure sensor 65 to thereby enable the determination of various parameters of blow-pressure measurements according to the oscillometric technique.

It will be appreciated that whereas pump 70 is illustrated as being manually-actuated, it could be automatically actuated to produce a precise control of the pressurization and depressurization of the air-sealed chamber 63.

Other techniques are known to provide a continuous measurement of blood pressure, and various parameters thereof, by controllably pressurizing an artery. One such other technique is the “Penaz” technique, as described for example in Penaz U.S. Pat. No. 4,869,261, the contents of which are incorporated herein by reference. According to this method, the external pump 70 would be controlled so as to cause the externally-applied pressure to be equal to the arterial blood pressure at all times, and the pressure used for this purpose would be measured by the pressure sensor 65.

The probe illustrated in FIG. 6, as well as those illustrated in FIGS. 1-5, may also be used for measuring various cardiac activities including heart rate, as well as various respiratory activities and other physical movements of the subject's body. For example, in the ear probe illustrated in FIG. 6, the overpressure produced by pump 70 could be set to about diastole, whereupon its actual value would depend upon the respiratory or other movements of the subject's body, such as respiration, walking or running, e.g., in order to measure distances an/or velocities traveled by the subject.

The probe may also be used during the nighttime to monitor and record not only cardiac activity, but also respiratory activity of the subject, in order to detect sleep apnea, snoring, etc. For the latter purpose, the ear probe illustrated in FIG. 6 includes a small speaker or buzzer 79 such that, when snoring is detected, the speaker or buzzer may be activated to produce a sound effective to stimulate the subject by biofeedback, to change position such as to cease snoring. Such a sound feedback is received only by the subject involved, and therefore does not disturb another person.

The illustrated ear probe may include a removable memory unit to monitor and record all cardiac and respiratory activities, body movements, sleep apnea, snoring, etc. of the subject during the nighttime for later evaluation. The electrical control and measuring system may be wire-connected to the ear probe, or may communicate therewith in a wireless manner as known in the art.

While the invention has been described with respect to several preferred embodiments, it will be appreciated that these are set forth merely for purposes of example, and that many other variations, modifications and applications of the invention may be made.

What is claimed is:

1. An ear probe for detecting and/or measuring at least one predetermined physiological condition of a subject, comprising:
an earplug shaped and dimensioned for insertion into an ear of the subject;
and a sensor carried by said earplug for sensing said predetermined physiological condition of the subject; said sensor including:
an acoustical transmitter and an acoustical receiver spaced from the transmitter and defining an acoustical channel therebetween;
and a processor for monitoring changes in the transit time, caused by said predetermined physiological condition, of an acoustical wave transmitted from said transmitter to said receiver through said acoustical channel.

2. The ear probe according to claim 1, wherein said processor monitors changes in said transit time by changing the frequency of the transmitter to produce and maintain a whole integer number of waves in a loop including said acoustical channel irrespective of changes in said physiological condition.

3. The ear probe according to claim 2, wherein said processor controls the frequency of said transmitter, to produce and maintain a whole integer number of waves in said acoustical channel, by detecting a fiducial point of each wave received by the receiver, and utilizing said detected fiducial point to trigger the transmission of the next wave by the transmitter.

4. The ear probe according to claim 3, wherein said processor utilizes the changes in frequency of the transmitter to produce a measurement of the respective physiological condition.

5. The ear probe according to claim 1, wherein said measured physiological condition is the ear temperature of the subject.

6. The ear probe according to claim 1, wherein said measured physiological condition is a cardiac condition of the subject as sensed by the pressure in the subject's carotid artery.

7. The ear probe according to claim 6, wherein said cardiac condition is heart rate, systolic pressure, diastolic pressure, mean arterial pressure, continuous blood pressure, stroke volume, or cardiac output of the subject.

8. The ear probe according to claim 1, wherein said measured physiological condition is a respiratory or movement condition of the subject as sensed by a physical movement in or of the subject's body.

9. The ear probe according to claim 8, wherein said respiratory or movement condition is respiration rate, respiration volume, sleep apnea, snoring, walking, or running of the subject.

10. The ear probe according to claim 8, wherein said respiratory or movement condition includes snoring by the subject; and wherein said ear probe further includes a speaker for producing an audio output upon detecting snoring by the subject such as to stimulate the subject by biofeedback to terminate the snoring.

11. The ear probe according to claim 1, wherein the earplug includes at least two of said sensors for measuring at least two physiological conditions of the subject.

12. The ear probe according to claim 11, wherein at least one of the said sensors measures the ear temperature of the subject, and the other of said sensors measures a cardiac, respiratory, or movement condition of the subject.

13. The ear probe according to claim 1, wherein said earplug is of a flexible material shaped and dimensioned to seal the ear canal and to produce a sealed air chamber between the end of the earplug and the subject's ear-drum; said sensors being exposed to said sealed chamber for sensing a condition therein.

14. The ear probe according to claim 13, wherein one of said sensors is a temperature sensor and measures changes in the temperature of said sealed air chamber as reflected by changes in temperature of the blood in the subject's carotid artery.

15. The ear probe according to claim 14, wherein the other of said sensors is a pressure sensor and measures changes in the pressure of said sealed air chamber as reflected by the blood pressure in the subject's carotid artery, by respiratory activity of the subject, or by movement activity of the subject.

16. The ear probe according to claim 15, wherein said acoustical channel in said pressure sensor is a body of an elastomeric material in which said transmitter and receiver are embedded at a predetermined spaced distance from each other, said body of elastomeric material being compressed by the pressure in said sealed air chamber to change the transit distance between the transmitter and receiver in response to changes in the pressure in said sealed air chamber.

17. The ear probe according to claim 15, wherein said acoustical channel in said pressure sensor, including said transmitter and receiver thereof, are carried by a deformable partition extending through a pressure-sensing chamber having one side communicating with said sealed air chamber such that said partition is deformable by the differential pressure on its opposite sides to thereby change the transit time between the transmitter and receiver in response to changes in the pressure in said sealed chamber.

18. The ear probe according to claim 17, wherein said ear probe further comprises an external source of pressure communicating with the other side of said pressure-sensing chamber.

19. The ear probe according to claim 18, wherein said external source of pressure is manually operable for initializing the pressure in said pressure-sensing chamber on the opposite sides of said deformable partition.

20. The ear probe according to claim 15, wherein said pressure sensor includes a passageway communicating with said sealed air chamber to controllably pressurize said sealed air chamber in order to utilize the probe for measuring the blood pressure of the subject.

21. The ear probe according to claim 13, wherein said earplug includes two of said sensors for measuring two physiological conditions of the subject.

22. The ear probe according to claim 21, wherein one of said sensors is exposed to and measures changes in the temperature of said sealed air chamber as reflected by changes in temperature of the blood in the subject's carotid artery, and the other of said sensors is exposed to and measures changes in the pressure in said sealed air chamber as reflected by changes in blood pressure in the subject's carotid artery, or by respiratory or movement activities of the subject.

23. An ear probe for detecting and/or measuring a plurality of physiological conditions of a subject comprising:
an earplug shaped and dimensioned for insertion into the ear canal of the subject;
a temperature sensor carried by said earplug for sensing the temperature of the blood in the subject's carotid artery;
and a pressure sensor carried by said earplug for sensing changes in blood pressure in the subject's carotid artery.

24. The ear probe according to claim 23, wherein said earplug is of a flexible material shaped and dimensioned to seal the ear canal and to produce a sealed air chamber between the end of the earplug and the subject's ear-drum; said temperature sensor being exposed to said air chamber to sense the temperature thereof; said pressure sensor being exposed to said sealed air chamber to sense changes in the pressure therein.

25. The ear probe according to claim 24, wherein said pressure sensor includes a body of elastomeric material which is compressed by the pressure in said sealed air chamber.

26. The ear probe according to claim 24, wherein said pressure sensor includes a membrane which is deformed by the pressure in said sealed air chamber.

27. The ear probe according to claim 24, wherein said ear probe further comprises an external source of pressure for initially pressurizing said pressure sensor.

28. The ear probe according to claim 24, wherein said ear probe comprises an external source of pressure for controllably pressurizing said sealed chamber, and thereby the subject's carotid artery, in order to utilize the probe for measuring the blood pressure of the subject.

29. The ear probe according to claim 24, wherein said pressure sensor senses a cardiac condition of the subject selected from the group consisting of heart rate, systolic pressure, diastolic pressure, mean arterial pressure, continuous blood pressure, stroke volume, and cardiac output.

30. The ear probe according to claim 24, wherein said pressure sensor senses a respiratory or movement condition of the subject selected from the group consisting of respiration rate, respiration volume, sleep apnea, snoring, walking or running movements.

31. The ear probe according to claim 24, wherein said pressure sensor senses snoring by the subject, and said ear probe further includes a speaker for producing an audio output upon detecting snoring by the subject such as to stimulate the subject by biofeedback to terminate the snoring.

32. The ear probe according to claim 24, wherein each of said sensors includes an acoustical transmitter and an acoustical receiver spaced therefrom and defining an acoustical channel therebetween; and wherein said ear probe further includes a processor for measuring changes in the transit time, caused by the respective physiological condition, of an acoustical wave transmitted from said transmitter to said receiver through the respective acoustical channel.

33. The ear probe according to claim 32, wherein said processor measures changes in said transit time in each sensor by controlling the frequency of the transmitter in the respective sensor to produce and maintain a whole integer number of waves in the respective acoustical channel irrespective of changes in the physiological condition measured by the respective sensor.

34. The ear probe according to claim 33, wherein said processor controls the frequency of said transmitter in each sensor to produce and maintain a whole integer number of waves in the respective acoustical channel by detecting a fiducial point of each wave received by the respective receiver, and utilizing said detected fiducial point to trigger the transmission of the next wave by the respective transmitter.

35. The ear probe according to claim 34, wherein said processor utilizes the changes in frequency of the transmitter in each sensor to produce a measurement of the respective physiological condition.
